(19) United States
(12) Patent Application Publication
(10) Pub. No.: US 2003/0143110 A1
(43) Pub. Date: Jul. 31, 2003

(54) Disinfection

(75) Inventors: Steven Kritzler, Cronulla (AU); Alex Sava, Paddington (AU)

Correspondence Address: LEYDIG VOIT & MAYER, LTD, 700 THIRTEENTH ST. NW, SUITE 300, WASHINGTON, DC, 20005-3960, US

(73) Assignee: Novapharm Research (Australia) Pty. Ltd., Rosebery (AU)

(21) Appl. No.: 10/151,139

(22) Filed: May 21, 2002

Related U.S. Application Data
(63) Continuation of application No. 09/720,330, filed on Feb. 22, 2001.

(30) Foreign Application Priority Data
Jun. 23, 1998 (AU) PP4273
Jun. 22, 1999 (WO) PCT/AU99/00505

(51) Int. Cl. A61L 2/22

(52) U.S. Cl. 422/29; 422/20; 422/292; 422/306

(57) ABSTRACT

The invention relates to a method of disinfection comprising the steps of sonicating a liquid disinfectant at a frequency selected to be above 15 MHz, preferably above 2 MHz in a nebulizing chamber to produce a nebulized disinfectant product. The frequency of the ultrasonic energy and the formulation of the disinfectant to which the ultrasonic energy is applied is such that 90% of microdroplets are between 0.8 and 2.0 micrometres in diameter. In preferred embodiments, the microdroplets are activated by the ultrasound and are substantially more effective than non-sonicated disinfectant. The invention also relates to compositions suitable for use in such methods which mar include activatable agents, surfactarits and/or agents to assist in drying.

### TECHNICAL FIELD

The invention relates to the field of disinfection.

### BACKGROUND

The disinfection of surfaces, for example of skin, non-autoclavable medical instruments, hospital wards, operating theatres, walls, hand rails, air conditioning ducts and the like remains one of the most problematic areas of infection control.

The majority of disinfection methods rely on direct contact of the surface to be disinfected with a liquid disinfectant These methods require considerable quantities of liquid disinfectants to ensure that all areas of the treated surface are covered with the disinfectant Usually the disinfectant is applied either as a liquid or a spray. Commonly the amount of disinfectant used is 100-100,000 times more than required to kill the microorganisms present on the surface. For example, 10⁻⁵ (0.00001) g of iodine is sufficient to kill all bacteria on a surface area of 1 m²with a contamination level of 10⁵ cfu/cm² in 10 minutes (Block, S. S., Disinfection, Sterlisation and Preservation, 3rd Edition, p.183) whilst the recommended amount of disinfectant would contain 0.1-0.2 g (10,000 times the level) of iodine. Such a high usage creates a series of problems with respect to cost, occupational safety and environmental impact.

Another problem associated with the traditional methods of contacting surfaces with liquid disinfectants is that of human toxicity. The use of disinfecting fluids which can be safely and conveniently handled by humans requires that the active disinfecting agents are typically present at low concentrations, resulting in unacceptably long contact times to achieve the required levels of disinfection.

For example, a commonly used aqueous disinfecting solution, containing 2% glutaraldehyde, requires soaking times of around 6 to 10 hours to achieve total kill.

Further problems may also be encountered when liquid disinfectants are applied to common surfaces, like walls, hand rails, air conditioning ducts and some bulky medical instruments. Apart from the stated practical difficulties in covering such surfaces with an even layer of the disinfectant, the surfaces usually contain minute cracks, crevices, and pores which can harbour bacteria. As the surface tension of most liquid disinfectants is relatively high, such areas are not penetrated and remain contaminated even after prolonged disinfection cycles.

One solution to the problem is the use of disinfectants in the gaseous phase which addresses the problem of access to cracks, crevices and pores. The small particle size of gaseous disinfectants creates another problem; the concentrations of the active biocidal chemicals need to be very high or the chemicals required are toxic and dangerous to handle. Several method employing disinfectants in the gaseous phase have been developed. The most common utilise either ethylene oxide and its analogues, or formaldehyde. Both compounds are extremely toxic, and have been identified as primary carcinogens. In addition, sterilising with the above gases requires a thorough control of pressure and humidity in the chamber, which necessitates the use of complex and expensive equipment. Thus, their use is limited to hospitals and critical medical instruments and requires careful supervision.

Another approach is used in a variety of plasma disinfecting methods. In these methods disinfection under essentially dry conditions is achieved using various active radicals and ions as the biocide. These can be formed from conventional disinfectants (as precursors) under plasma forming conditions. In addition to the cost and complexity of plasma equipment, these methods tend to result in degradation of many construction materials such as are used in endoscopes and other instruments. Obviously, plasma methods can not be used for bulky equipment and large surfaces.

An area of particularly difficulty is in the field of dentistry and dental prosthetics.

The invention will be described herein with particular reference to its use in that field but it will be understood not to be limited to that use.

Dental personnel are exposed to a wide variety of pathogens in the blood and saliva of patients. These pathogens can cause infections such as the common cold, pneumonia, tuberculosis, herpes, viral hepatitis and HIV.

A particular problem occurs when contaminated dental impressions taken from patients' mouths are used to make dental casts. In these circumstances, microorganisms from the impression material are transferred to the cast. This infected cast can, in turn, contaminate the pumice pans and polishing wheels which are used in shaping the casts for manufacturing prosthetic devices. This shaping procedure, in turn, produces an atmosphere of infectious dust which is potentially hail. The polishing of dentures with a common pumice pan and polishing wheel can lead to cross-contamination between patients.

Disinfection of the impressions and casts has been recommended as a method of preventing the transfer of infection in the field of dental prosthetics. The most commonly used impression materials are alginate-based. Alginates tend to swell on soaking in aqueous solutions, thus reducing the accuracy of the subsequently derived casting and ultimately, resulting in an unsuitable prosthetic device.

To overcome the immersion of alginates into bulk liquids, a number of researchers recommend using spray atomised disinfectants generated by manual spray pumps.

When spray atomised disinfectants are used, a considerably smaller amount of liquid is brought into contact with the impression than is the case with immersion and thus the potential liquid absorption is reduced. However the shape of the dental impression is complex and it requires spraying from different angles to achieve even coverage. Thus the amount of disinfectant delivered into the contact with alginate is sufficient to distort the alginate by additional swelling while being insufficient to ensure even coverage of the surface.

A number of studies have shown that the efficacy of registered disinfectants when used as a spray to coat a very uneven surface is low. See for example “Efficacy of Various Spray Disinfectants on Irreversible Hydrocolloid Impressions”; Westerholm, Bradley, Schwartz—Int J Prosthodont 1992;5:47-54). 5.25% sodium hypochlorite and 2% glutaraldehyde achieve only a log 3 to log 4 reduction in a bacterial population of Staphylococcus aureus and M. phlei when sprayed on to the alginate impressions. These liquids, which are expected to be highly efficacious, achieve only a log 2 reduction in the number of microbial pathogens when they were sprayed on impressions inoculated with vegetative Bacillus subtilis. A severe disadvantage of the various spray methods is the probability of severe irritation to eyes and mucous membranes by the atomised liquid disinfectants.

Methods of atomising liquids using ultrasonic irradiation have been cited in previous art for atomising liquid medicine, disinfectants and for moisturising human tissues. For example, U.S. Pat. No. 4,679,551 discloses the use of a low frequency ultrasonic sprayer for moisturising the oral cavity of terminal patients. Igusa et al U.S. Pat. No. 5,449,502 describes the use of an ultrasonic transducer vibrating at 30-80 kHz to atomise a disinfecting solution and deliver a sufficient amount of the solution for the disinfection of hands. WO 97/17933 discloses a method of spraying liquids onto human tissue using sprays produced by low frequency (20 to 200 kHz, preferably 20-40kHz) ultrasonic irradiation utilising a spray gun described in U.S. Pat. No. 5,076,266. The atomisation at low frequency produces, in large part, particles with diameters in the range of 5 to 10 micrometers. This is of the same order or larger than that obtained by the application of mechanical spraying techniques. As a result, the amount of liquid accumulating on the treated surface is significant. This amount of liquid is sufficient to cause unacceptable dimensional distortion of moisture sensitive materials such as dental alginate impressions.

Low frequency (ie 40 KHz) ultrasonic irradiation has been recognised as a means of quantitatively transferring bacteria from solid surfaces (eg AOAC Method of Analysis No. 991.47) and thus is not of itself bactericidal.

U.S. Pat. No. 4,298,068 discloses apparatus for sterilization of food containers in which a sterilization agent is heated and atomized. Ultrasound may optionally be used to generate the mist Frequencies of 30-100 KHz and 1.0-2.0 MHz are disclosed. Both are said to produce droplets of 2.0-5.0 microns at 50-80° C. The method, while providing a reduction in bacterial contamination, does not provide sterilization at acceptable cost

U.S. Pat. No. 4,366,125 discloses apparatus for sterilizing sheet material with hydrogen peroxide utilizing a combination of ultrasound to generate a treating mist in combination with UV irradiation of the sheet downstream of the peroxide treatment. The ultrasound is at 1-2 MHz and produces droplets of which most are aprox 10 micron diameter. Significantly, sterilization with UV followed by treatment with peroxide was ineffective. Also substituting immersion of the material to be treated in peroxide was of similar effectiveness to using ultrasound generated mist. This method has the disadvantage of involving substantial capital and running costs for the UV line, and is not applicable to treat non sheet material having internal surfaces which would be shadowed from UV.

U.S. Pat. No. 4,680,163 discloses a method for sterilizing non conductive containers by generating a mist of sterilizing agent with ultrasound and electrically charging the droplets by means of a corona discharge. The charged droplets are deposited on the wall of the container under the influence of the electric field. The ultrasound frequency is 1-5 MHz (although only 1.75 MHz is exemplified). Mist droplets of diameter less than 10 micron, preferably in the range of 2-4 micron, are generated. The container must be surrounded by a high voltage electrode. The corona discharge is said to decompose the peroxide to form atomic oxygen. The method suffers form the disadvantage that the high voltages employed (20-50 kV) raise safety concerns due to the risks of electrocution or ozone poisoning and the degree of sterilization obtainable is less than desired. Moreover the method is of limited applicability in view of the need to surround the surface to be treated by a high voltage electrode.

None of the methods employing ultrasound is suitable for disinfection of skin, hollow medical instruments hospital surfaces or the like

It is an object of the present invention to overcome or ameliorate one or more of the disadvantages of the prior art, or at least to provide a useful alternative.

### SUMMARY OF THE INVENTION

According to a first aspect, the invention consists in a method of disinfection comprising the step of applying ultrasound energy at a frequency selected to be above 1.5 MHz to a liquid composition comprising a disinfectant in combination with at least one surfactant, to produce a nebulised disinfectant product.

Preferably the frequency of the ultrasonic energy and the liquid disinfectant formulation (including surfactant) are selected such that 90% of microdroplets are between 0.8 and 2.0 micrometres in diameter.

The applicant has found that when a disinfectant is combined with a surfactant and then atomised by an ultrasonic nebuliser at frequencies greater than 1.5 MHz, a reduction in particle size of the nebulized product is obtainable in comparison with the particle size obtained in the absence of the surfactant at the same frequency, and significantly improved disinfection is obtained in comparison with immersion or with sprays of the same or similar disinfectants, including sprays nebulised at lower frequencies. Without wishing to be bound by theory, it is believed that the improvement is due to activation of the disinfectant by ultrasonic irradiation at the selected frequency and not merely to smaller particle size.

The droplets of the atomised disinfectant containing the activated biocidal compound are desirably delivered onto the surface to be disinfected as a cold (preferably below 40° C.) mist of microdroplets.

The amount of disinfectant delivered, the concentration of the disinfectant mist and condensation conditions are regulated by selection of the quantity and type of surfactant incorporated, by varying the size of the droplets, the air flow conditions and the period of disinfectant contact with the surface to be disinfected.

Preferably, the nebulising time and ultrasonic frequency are selected in combination having regard to the disinfectant composition to provide a predetermined level of disinfection of an object exposed to the nebulised product.

The surfaces to be disinfected may be for example skin, medical instruments, hospital wards, operation theatres, walls, hand rails, air conditioning ducts, dental and medical prosthesis, skin, and open wounds but are not limited to such surfaces.

The present invention also relates to the disinfection of a volume contained within an enclosed space.

According to a second aspect of the invention the size of microdroplets and their susceptibility to activation is modified by the addition of a surfactant or surfactant system. A “surfactant” as herein defined is any surface active agent, that is to say any composition which alone or in combination with other substances acts to reduce the surface tension of the disinfectant. A consequence of reduced surface tension may be an increase in vapour pressure of the disinfectant composition. Suitable surfactants include alcohols, ethoxylated alcohols, wetting agents and other surface active agents.

Preferably the disinfectants selected for use in the present invention are compounds which can be activated by, high frequency ultrasound. Disinfectants useful in the present invention include, but are not limited to, those which improve their performance when exposed to high frequency ultrasonic irradiation, for example those based on the peroxy compounds (e.g. hydrogen peroxide, peracetic acid, persulphates and percarbonates), halogen solutions, halogen compounds and solutions of halogen compounds (e.g. sodium hypochlorite and povidone iodine), phenolic compounds and halogenated phenolic compounds in solution (e.g. Triclosan) have been found to benefit from ultrasonic irradiation.

According to a third aspect the invention consists in performing the disinfection within an enclosed disinfection chamber, such that nebulisation occurs in a nebulising chamber which resides in or communicates with the enclosed disinfection chamber.

According to a fourth aspect, the invention consists in a method according to the first or second aspects further comprising the step of nebulizing one or more neutralising agents, for example peroxidase enzymes for peroxy-compounds or sodium thiosulfate for halogen based disinfectants, after the completion of a sterilisation cycle to decompose all active biocides.

According to a fifth aspect, the invention consists in selecting a combination of nebulising time and ultrasonic frequency having regard to the disinfectant composition so as to ensure adequate disinfection of a predetermined object. Preferably the nebulising time and ultrasonic frequency are selected such that disinfection occurs with a minimum of liquid and such that the disinfected object is quickly and easily dried. This can be achieved by air drying, blow drying or vacuum or by a combination of these, whereby a given level of sterilisation and drying of an object may be achieved in a minimum time at ambient temperature.

According to a sixth aspect, the invention consists in a disinfected volume in a nebulising chamber prepared according to one of the methods of the invention.

The invention also consist in a method of disinfection comprising the step of nebulising a liquid disinfectant composition including at least one surfactant to form microdroplets, allowing the microdroplets to contact a surface and applying ultrasonic energy to at least one of the surface and the microdroplets.

The invention further consists in a mist of droplets of which a majority have a particle size of below 2 microns in diameter and comprising a disinfectant in combination with a surfactant for use in accordance with the methods of the invention.

Unless the context clearly requires otherwise, throughout the description and the claims, the words ‘comprise’, ‘comprising’, and the like are to be construed in an inclusive as opposed to an exclusive or exhaustive sense; that is to say, in the sense of “including, but not limited to”.

### BRIEF DESCRIPTION OF THE DRAWINGS

FIG. 1 shows an embodiment of a disinfection apparatus in accordance with one aspect of the present invention.

FIG. 2 shows a preferred configuration of an embodiment of a disinfection apparatus in accordance with one aspect of the present invention.

FIG. 3 shows another preferred configuration of an embodiment of a disinfection apparatus in accordance with one aspect of the present invention.

### BEST MODE FOR CARRYING OUT THE INVENTION

The invention will now be described by way of example only with reference to preferred embodiments.

Ultrasonic and acoustic vibrations are known to produce aerosols. The mechanism of atomising liquids with ultrasound consists of the microeruption of cavitation bubbles close to the liquid/air interface: breaking bubbles scatter the liquid. Using air flows generated either by pumping air or by the Bernoulli effect, the mist of droplets can be separated from the bulk of the liquid and directed onto an object.

The invention will be described with particular reference to its use with hydrogen peroxide based disinfectants but it will be understood not to be limited to these disinfectants.

It is believed that the mode of biocidal action of commonly used disinfectants is not due to the molecule itself, but to the production of more powerful derivatives, for example, the hydroxyl radical in the case of peroxy compounds or hypochlorous acid in the case of hypochlorite-based disinfectants. These radicals normally form as a result of irradiation with ultraviolet or infrared radiation or the catalytic action of metal ions.

Hydrogen peroxide vapour sterilisers have been used in the past. These sterilisers have a series of drawbacks, amongst which is the need for a high temperature to generate vapour. The increased temperatures are required for vaporisation and the production of active biocidal particles. As the concentration of hydroxyl radicals is directly proportional to the concentration of hydrogen peroxide in the formulation and the temperature, the highest practical temperature and concentration are used.

In the present invention high frequency ultrasonic energy is utilised for both the atomisation of disinfectant solutions and the production of biocidally active hydroxyl radicals. The presence of at least one surfactant has been found to mediate a significant reduction in particle size, and a significant increase in activation of the disinfectant allowing achievement of the required concentrations of biocidal actives without increasing the temperature or the concentration of biocide in the bulk liquid.

The combination of atomisation and activation by ultrasound in the presence of one or more surfactants overcomes the major drawbacks of the previous art. The amount of antiseptic vapour delivered on the object to be disinfected is very much less than required for bulk liquid and spray disinfection methods. The particle size of less than 2.0 micrometres, (preferably 0.8-2.0 micrometers), of the majority of the atomised mist is of the same order as the size of the smallest cracks and pores which can potentially harbour microorganisms.

The layer of the condensed antiseptic which forms in the course of, and subsequent to, sonication contains a sufficient amount of active biocide to destroy all susceptible microorganisms.

The low concentration of disinfectant, in the case of hydrogen peroxide, left on the disinfected object rapidly decomposes forming harmless water and oxygen. If the remaining peroxide needs to be decomposed after treatment, a small amount of peroxidase enzymes or any other suitable neutraliser can be atomised on the object.

In the case of other disinfectants the small amounts remaining on the surface can be left, neutralised or rinsed off as required.

When subjected to ultrasound at 1.2 MHz water produces particles with the mass median aerodynamic diameter (MMAD) of 4-5 micrometres (The Ultrasonic Generation of Droplets for the production of Submicron Size Particles, Charuau, Tierce, Birocheau; J Aerosol Sci. V. 25, Suppl.1, ppS233-S234, 1994). At lower frequencies the particles are larger and at higher frequencies the MMAD is reduced. At 2.5 MHz, MMAD is 1.9 micrometres. Further increase in frequency results in the increase of energy density and hence an increase in the temperature of the nebulised liquid. The inventor has found that a further reduction in aerosol particle size to 0.8-1.0 micrometres can be achieved by decreasing the surface tension by the addition of a small amount of an appropriate surfactant without significant increase in temperature.

A mixture of water soluble surfactants with the addition of non-water soluble surfactants to suppress foam is found to be effective in one of the embodiments of the current invention.

Suitable surfactants can include a mixture of ethoxylated alcohols (eg Teric 12A3) together with dodecylbenzenesulfonic acid salts, or ethoxylated alcohols alone or block copolymers of ethylene oxide and propylene oxide with alcohol either alone or as part of a mixture with the above surfactants. A skilled addressee would understand that the above surfactants are included only as non-limiting examples of species which can be applied as part of the invention.

The amount of liquid condensed on a surface after a 2 minute exposure to nebulised droplets in a sealed system was found to be in the order of 30 g/m² for low frequency ultrasound. When ultrasound in the high frequency range which is the subject of this invention is used, the condensate level was found to be reduced to 3 g/m² in the same sealed system

A substantial advantage of the invention is associated with the small amount of condensate formed on surfaces. Inclusion in the disinfectant of substances with high vapour pressure is advantageous to reduce drying time. For example alcohols with high vapour pressure relative to water, ethers with high vapour pressure relative to water, hydrocarbons with high vapour pressure relative to water, esters with high vapour pressure relative to water and other organic substances with high vapour pressure relative to water or mixtures of such substances with high vapour pressure may substantially reduce the time required for drying.

Even when the disinfectant utilised in the process has a relatively high vapour pressure (eg aqueous hydrogen peroxide solution), this material can be easily removed by air drying. At a relative humidity of 50 to 60% and a temperature of 22° C. the air drying of an object with a surface area of 100 to 150 cm² is achieved in 10 to 15 minutes. However if warm, dry air is blown across the surface of the object the drying time is reduced to 0.5 to 3 minutes. Therefore a high speed, cold disinfection cycle which begins with a microbially contaminated instrument and results in a dry, disinfected instrument can be achieved quickly, simlply and cheaply.

The application of such equipment is potentially very broad and includes hospitals, medical clinics, dental clinics, veterinary clinics, food processors, fast food outlets, beauty salons, hairdressers, tattoo parlours, etc.

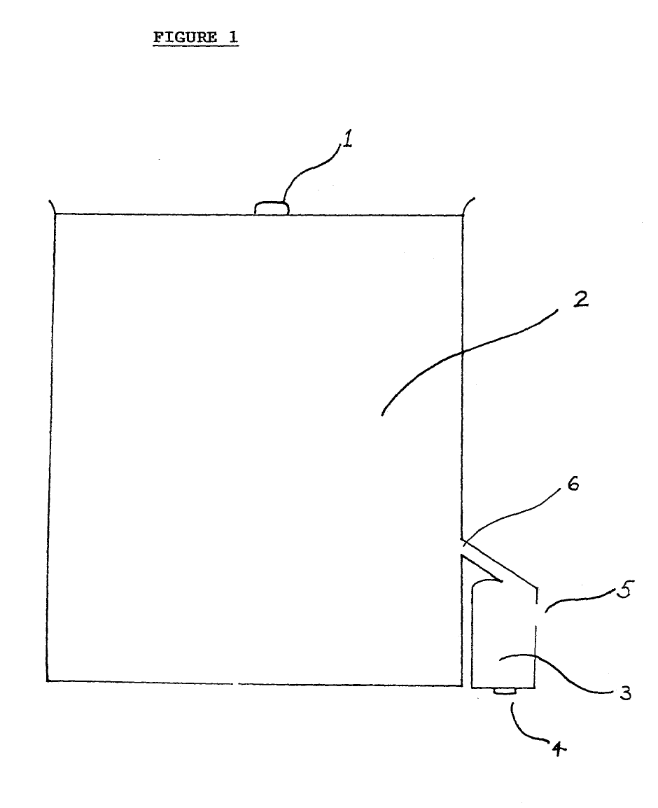

With reference to the drawings, FIG. 1 shows an embodiment of a disinfection apparatus suitable for use in the present invention. An article to be disinfected is placed in enclosed chamber 2. The lid of the chamber 1 is removable for this purpose. The disinfectant is placed in ultrasonic nebulising chamber 3, and nebulised by ultrasonic transducer 4. The nebulizer intake 5 provides the necessary air from outside the chamber. Nebulized disinfectant produced in nebulizing chamber 3 enters disinfection chamber 1 via an outlet 6. Preferably outlet 6 comprises a tube disposed at an angle to the direction of sonication whereby to minimize entrainment of large drops if any.

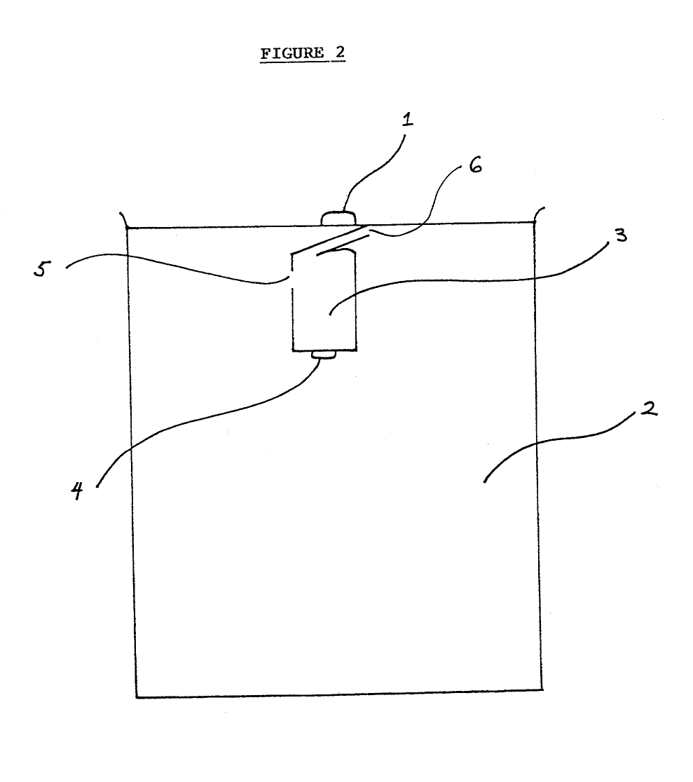

FIG. 2 shows a preferred embodiment of a disinfection apparatus suitable for use in the present invention. An article to be disinfected is placed in enclosed chamber 2 by means of a removable lid 1. The disinfectant is placed in ultrasonic nebulising chamber 3 and nebulised by ultrasonic transducer 4. The nebuliser intake 5 provides the necessary air from inside the chamber.

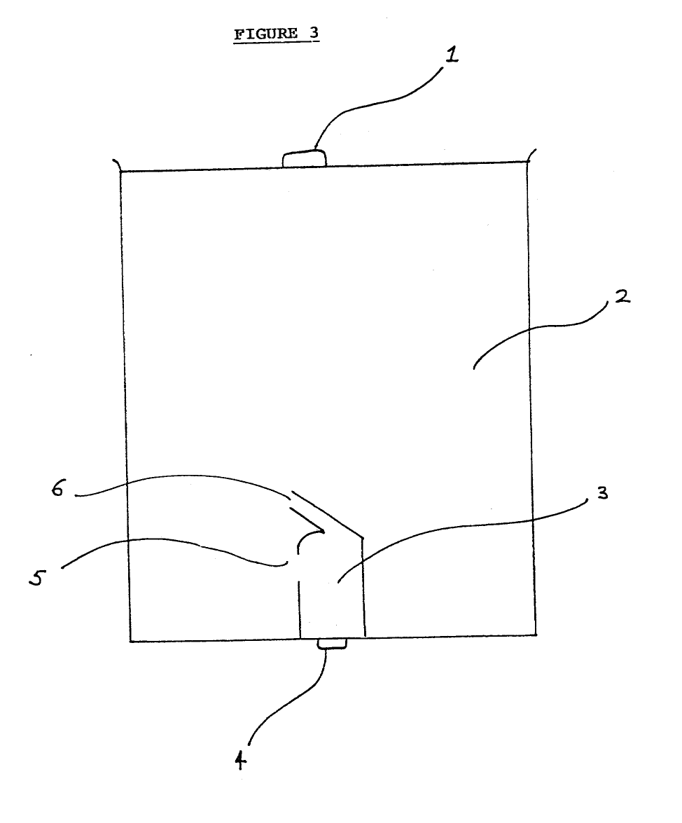

FIG. 3 shows an adaptation of the apparatus according to FIG. 2. While ultrasonic transducer 4 is located outside the chamber, nebuliser intake 5 still provides the necessary air from within the enclosed chamber 2.

The advantage of configurations shown in FIGS. 2 and 3, and similar configurations is that they provide a completely sealed system. The disinfectant both prior to, and after, nebulisation is contained within the sealed system, providing significant advantages over unsealed systems where the disinfectant has implications with respect to human health and safety.

In the embodiments of FIGS. 2 and 3, when the transducer is energized, nebulized disinfectant from nebulization chamber 3 within sealed disinfection chamber 1 directly enters chamber 1 via nebulizer outlet 6. Consequently, the concentration of nebulized disinfectant in the sterilisation chamber 1 increases and air entering intake 5 from sealed chamber 1 carries an increasing concentration of nebulized disinfectant which is thus recycled.

Embodiments of the invention will now be exemplified.

### EXAMPLE 1

Efficacy data was obtained with the following disinfectants:

A. 6% w/w hydrogen peroxide (pH=3), 94% w/w water.

B. 6% w/w hydrogen peroxide+15% w/w n-propanol+0.3% w/w Irgasan DP300+0.02% w/w PVP K15+0.5% w/w STPP (pH=7)+2% w/w LAS+2% w/w Teric12A3

C. 5% w/w peroxyacetic acid, diluted 1:50 with distilled water

D. 2% w/w chlorhexidine gluconate+15% w/w n-propanol in distilled water

Test Procedures:

Equipment. The principle of operation of nebulisers is described elsewhere, (for example by K. Sollner in Trans. Farady Soc. v.32, p1532, 1936). The main elements of an ultrasonic nebuliser are: a high-frequency generator, a piezoceramic transducer and a reservoir for the solution to be nebulised. The production of a fine aerosol involves forcing the transducer to vibrate mechanically by applying resonance frequency. These high frequency vibrations are focussed in the near surface part of the solution, and create an “ultrasonic fountain”

Once the energy exceeds a certain threshold, droplets break off and are forced by air streams out of the reservoir.

A Mousson 1 ultrasonic nebuliser (currently discontinued, similar nebulisers are manufactured by Otto Schill GmbH & Co., K. Medizintechnik, Germany) with a concave glass covered transducer was used to atomise the various disinfectants under study. The nebuliser operates at 2.64 MHz. The nebulising rate was approximately 1 mL/min. The nebulised liquid disinfectant was pumped into a 1.5 L hermetically sealed vessel (FIG. 1) for 2 minutes. Normally the disinfectant vapour pressure in the vessel reaches the same value as in the nebulising chamber of the nebuliser within 30-40 seconds. As the nebulising rate depends on the pressure differential, the vapour delivery rate reduced significantly after 30-40 seconds, and was just sufficient to compensate for the condensed vapour. Total amount of nebulised disinfectant during the cycle was under 1 mL.

The inoculated carriers were placed in the close vicinity of the nebulising horn.

Inoculum:

The inocula of vegetative Pseudomonas aeruginosa (ATCC15442), Mycobacterium terrae (ATCC 15755), E.coli (ATCC 8739), and S.aureus (ATCC 6538), were prepared from an overnight culture and contained approximately 10⁸-10⁹ cfu/mL.

The inoculum of dry, non vegetative Clostridium sporogenous (ATCC 3584), and B.subtilis (ATCC 19659) spores was prepared as per the method described in AOAC 966.04.

Each carrier was inoculated with approximately 0.02 mL of the inoculum to provide for contamination levels of 10⁶-10⁷ cfu per carrier.

Carriers:

20 microlitres of an inoculum was placed on sterile (3 hours at 180 C. oven) 10×20 mm glass plates, ad dried for 40 minutes in the incubator at 36° C. Sterile (3 hrs at 180° C.) glass penicylinders were soaked in the inoculum for 10 minutes and then for 40 minutes in the incubator at 36° C.

Alginate slices were prepared from Fast Set Alginate powder (Palgat Plus Quick, ESPE) sterilised for 1 hr at 120° C. The alginate was hand mixed for 30 seconds using manufacturer recommended water/powder ratio and loaded onto dry sterile trays. After settling for 3 minutes alginate has been cut with a flame-sterilised scalpel into a 20×10×1 mm slices. The slices there aseptically placed on a sterile Petri dish and contaminated by pressing the scalpel soaked in inoculum onto the slices. Extreme care was taken to avoid inoculation of the slides sand the surface of Petri dish.

Sterile silicone slices were prepared from Hydrophilic Vinyl Polysiloxane Impression Material (Heavy Body, Normal Setting, ADA Spec. 19, Elite H-D by Zhermack) using mixing procedure recommended by the manufacturer and loaded onto a sterile tray. After setting for five minutes, the impression material was cut into a 20×10×1 mm slices with the sterile scalpel. The slices were sterilised by soaking in a 1% peroxyacetic acid for three minutes, then rinsed with the sterile water and dried under UV light for five minutes. The slices were aseptically placed on a sterile Petri dish and contaminated by pressing the scalpel soaked in inoculum onto the slices.

A Petri dish with inoculated carriers was placed into the disinfecting vessel. The vessel was then covered tightly with a lid to ensure that nebulised liquid could not escape from the vessel. The disinfection cycle consisted of 2 minutes nebulising, and then left for four minutes to allow the vapour to condense.

Immediately after opening the lid, each carrier was aseptically placed in the test tube with sterile nutrient broth containing disinfectant deactivator (Tween 80). Bacto Letheen broth was used for P. aeruginosa, S. aureus and E.coli, a Bacto Middlebrook 7H9 both for M. terrae and a Bacto Fluid Thioglicolate Media for the spores. As a control, inoculated carriers were treated with nebulised, sterile distilled water in place of disinfectant

Essentially, this experiment is modelled on the AOAC's sterilant testing methods. No growth in the test tube indicates that 100% kill of a test organism has been achieved. This is a significantly more severe requirement than the log 5 reduction in the bacteria population required by the ADA. This method has been chosen as the surest method for demonstrating the efficacy of disinfecting techniques.

Results:

TABLE 1
Mycobacterium terrae:
Inoculum: 10⁸ cfu/mL in tryptone soya broth

Carrier/

| /disinfectant | A | B | C | D |
|---|---|---|---|---|
| Glass slides | passes | passes | passes | passes |
| Glass penicylinders | nt | passes | passes | nt |
| Silicone | nt | passes | passes | nt |
| Alginate slices | passes | passes | passes | growth 8 out of 8 |

“nt” - not tested
“passes” - complete kill of the tested organism has been achieved on at least 10 out of 10 replicas, with no survivals
“growth” number of carriers which carried viable test organisms

TABLE 2
Pseudomonas aeruginosa
Inoculum: 10^{8 cfu/mL in tryptone soya broth}

Carrier/

| /disinfectant | A | B | C | D |
|---|---|---|---|---|
| Glass slides | passes | passes | passes | passes |
| Glass | 5 out of 9 | passes | passes | growth 6 out of 10 |
| penicylinders | | | | |
| Silicone | nt | passes | passes | growth 10 out of |
| | | | | 10 |
| Alginate slices | growth | passes | passes | growth 9 out of 10 |
| | 8 out of 10 | | | |

TABLE 3
E.coli:
Inoculum: 10⁸ cfu/mL in tryptone soya broth

Carrier/

| disinfectant | A | B | C | D |
|---|---|---|---|---|
| Glass slides | passes | passes | passes | passes |
| Glass penicylinders | nt | passes | passes | nt |
| Silicone | nt | passes | passes | nt |
| Alginate slices | nt | passes | passes | nt |

TABLE 4
S.aureus:
Inoculum: 10⁸ cfu/mL in tryptone soya broth

Carrier/

| disinfectant | A | B | C | D |
|---|---|---|---|---|
| Glass slides | passes | passes | passes | passes |
| Glass penicylinders | growth 3 out of 10 | passes | passes | nt |
| Silicone | nt | passes | passes | nt |
| Alginate slices | nt | passes | passes | nt |

TABLE 5
Clostridium sporogenes dried spores:
Inoculum: 10⁸ cfu/mL in tryptone soya broth

Carrier/

| disinfectant | A | B | C | D |
|---|---|---|---|---|
| Glass slides | passes | growth 4 out of 10 | passes | passes |
| Glass penicylinders | nt | growth 5 out of 10 | passes | passes |
| Silicone | nt | nt | nt | nt |
| Alginate slices | nt | nt | nt | nt |

“nt” not tested
“passes” complete kill of the tested organism has been achieved on at least 10 out of 10 replicas, no survivals
“growth” number of carriers which carried viable test organisms

### EXAMPLE 2

Assessing the efficacy of the disinfectants on alginate dental impressions using a sealed system (FIG. 2).

The testing procedure has been adapted from that described in U.S. Pat. No. 5,624,636. Sterile models of a patient's maxillary and mandible teeth and soft tissues were contaminated with the bacterial suspensions containing 10⁸ to 10⁹ cfu/mL in sterile water. Fast set alginate dental impressions (Palgat Plus Quick, ESPE) were hand mixed for 30 seconds using the water/powder ratio the manufacturer recommended, and loaded onto sterilised plastic trays.

The impressions were made of contaminated models, and these were allowed to bench set for 3 minutes, after which time the models were removed. To transfer viable bacteria the parts of the impressions containing the 12th and 13th teeth (UL4 and UL5) for maxillary jaws and 30th and 29th (LL4 and LL5) teeth for the mandible jaws were cut out with a sterile scalpel and placed into 10 mL of sterile tryptone soya broth, sonicated in a 40 KHz ultrasonic bath for 2 minutes, plated onto tryptone soya agar and incubated aerobically for 48 hours. After disinfection, the parts of the impressions containing 4th and 5th (UR4 and UR5) teeth for maxillary jaws or 28th and 28th (LR4 and LR5) teeth for the mandible jaws were cut out and viable bacteria were transferred in the tryptone soya broth as described above. Both maxillary and mandible impressions were processed in the same cycle. The tabulated results of bacterial survivals are an average between the bacterial populations of the two impressions.

TABLE 6
Alginate impressions
Inoculum: Pseudomonas aeruginosa 10⁸ cfu/mL in tryptone soya broth

| | A | B | C | D |
|---|---|---|---|---|
| Before treatment, | 3 × 10⁷ | 3 × 10⁷ | 3 × 10⁷ | 3 × 10⁷ |
| cfu per impression | | | | |
| After treatment, | 1.2 × 10⁴ | 85 | 47 | 6.4 × 10³ |
| cfu per impression | | | | |

TABLE 7
Alginate impressions
Inoculum: Pseudomonas aeruginosa 10⁸ cfu/mL in tryptone soya water

| | A | B | C | D |
|---|---|---|---|---|
| Before treatment, | 4.5 × 10⁷ | 4.5 × 10⁷ | 4.5 × 10⁷ | 4.5 × 10⁷ |
| cfu/mL | | | | |
| After treatment, cfu/mL | 7.2 × 10³ | 0 | 0 | 4.3 × 10³ |

TABLE 8
Alginate impressions
Inoculum: E.coli 10⁸ cfu/mL in tryptone soya broth

| | A | B | C | D |
|---|---|---|---|---|
| Before treatment, | 8 × 10⁶ | 8 × 10⁶ | 8 × 10⁶ | 8 × 10⁶ |
| cfu/mL | | | | |
| After treatment, cfu/mL | 5.5 × 10² | 0 | 0 | 3 × 10⁴ |

TABLE 9
Alginate impressions
Inoculum: Pseudomonas aeruginosa 10⁸ cfu/mL
in tryptone soya broth, rinsed after
inoculation with 250 mL sterile tap water as per the ADA protocol

| | A | B | C | D |
|---|---|---|---|---|
| Before treatment, | 9 × 10⁴ | 9 × 10⁴ | 9 × 10⁴ | 9 × 10⁴ |
| cfu/mL | | | | |
| After treatment, cfu/mL | 0 | 0 | 0 | 60 |

### EXAMPLE 3

To compare the biocidal efficacy of sonicated and non-sonicated solutions of hydrogen peroxide the following experiment was conducted. 0.1 mL inocula of P.aeruginosa (10⁹ cfu/mL) and vegetative Bacillus subtilis were spread evenly over 20×15 mm areas of glass plates, dried for 40 min and then treated with 0.05 mL of 4% hydrogen peroxide for 2 minutes. The surviving microorganisms were transferred, as described in example 1, into tryptone soya broth and then plated. The same contaminated plates were treated for 15 seconds With the nebulised mist of the same 4% hydrogen peroxide solution, and then left for 1 minute and 45 seconds. The total amount of hydrogen peroxide condensed on each plate was below 0.01 mL (or at least 10 times less than in the reference experiment). The results were as follows: In the experiment with the bulk solution the observed survival level was 4×10³ cfu/mL; the nebulised hydrogen peroxide killed all bacteria and no survivors were detected either on Petri dishes, or in the test tubes with tryptone soya broth.

### EXAMPLE 4

A 1% hypochlorite disinfecting solution has been used to disinfect mandible dental impressions made of the same model as described in Example 2. Three different modes of disinfectant delivery were compared:

1. Atomised with a fine spray hand pump (AC Colmack Ltd). The disinfectant was sprayed on the impressions and left for 10 minutes.

2. Atomised with a 40 KHz Micronist ultrasonic atomiser (Misonix Inc) for 3 minutes, then left for another 8 minutes. Total contact time is 10 minutes.

3. Atomised with a 2.64 MHz Mousson ultrasonic nebuliser for three minutes and then left in the nebulising chamber (sealed system) for seven minutes. Total contact time is 10 minutes.

The results are as follows:

TABLE 10

| | Amount of | Contamination levels, | |
|---|---|---|---|
| | Disinfectant | cfu per impression | |
| Delivery Mode | Delivered | Before disinfection | After disinfection |
| Hand Sprayed | 0.41 g | 8.7 × 10⁷ | 3.9 × 10² |
| 40 kHz | | | |
| nebuliser | 0.28 g | 1.2 × 10⁷ | 2.4 × 10² |
| 2.6 MHz | | | |
| nebuliser | 0.06 g | 5.3 × 10⁷ | 0 |

It can be seen that greater kill levels are achieved when the mixture is nebulised at 2.6 MHz than by the other methods. The quantity of disinfectant used is also significantly lower

### EXAMPLE 5

Biocidal efficacy of sonicated disinfectants with and without surfactants was compared as follows.

Aqueous solutions:

| | |
|---|---|
| CL: | 0.5% sodium hypochlorite |
| CLA: | 0.5% sodium hypochlorite + 0.5% LAS |
| CLN: | 0.5% sodium hypochlorite + 0.5% PEG6200 (BASF) |
| HP: | 1% hydrogen peroxide |
| HPA: | 1% hydrogen peroxide + 0.5% LAS |
| HPN: | 1% hydrogen peroxide + 0.5% PEG6200 |
| HPE: | 1% hydrogen peroxide + 5% Ethanol |

were nebulised in the closed chamber (using Musson-1 2.64 MHz ultrasonic nebuliser) on glass plates with dried inoculum of P.aeroginosa (10⁹ cfu/mL) and vegetative Bacillus subtilis until evenly covered with the condensed nebula Then the glass plates were transferred, as described in example 1, into tryptone soya broth in order to quantify surviving microorganisms. The total amount of condensed disinfectant was weighed using an analytical balance and the time taken to evenly cover the plates with the nebula was noted.

The results are:

| | | Amount of | P. aeroginosa | | B. subtilis | |
|---|---|---|---|---|---|---|
| Disinfectant | time, sec | disinfectant, mg | Before | After | Before | After |
| CL | 100+/−10 | 80+/−20 | 6.5 * 10⁷ | 0 | 7.1 * 10⁶ | 1.4 * 10⁴ |
| CLA | 50+/−5 | 40+/−10 | 6.5 * 10⁷ | 0 | 7.1 * 10⁶ | 5.0 * 10¹ |
| CLN | 55+/−5 | 40+/−10 | 6.5 * 10⁷ | 0 | 7.1 * 10⁶ | 2.2 * 10¹ |
| HP | 110+/−8 | 100+/−10 | 6.5 * 10⁷ | 3.3 * 10³ | 7.1 * 10⁶ | 6.1 * 10² |
| HPA | 60+/−5 | 50+/−10 | 6.5 * 10⁷ | 0 | 7.1 * 10⁶ | 0 |
| HPN | 60+/−5 | 50+/−10 | 6.5 * 10⁷ | 0 | 7.1 * 10⁶ | 0 |
| HPE | 55+/−5 | 60+/−10 | 6.5 * 10⁷ | 0 | 7.1 * 10⁶ | 0 |

Thus, the nebulised disinfectants with reduced surface tension possess significantly better bactericidal properties. Not less than 90% of the droplets of modified surface tension disinfectants (CLA, CLN, HPA, HPN, HPE) had MMAD below 2.0 microns, whilst the MMAD of disinfectants (HP and CL) with non-modified surface tension was between 2.5 and 5 microns.

Although the invention has been described with reference to specific examples, it will be appreciated by those skilled in the art from the reading hereof that the invention may be embodied in other forms without departing from the scope of the concept herein disclosed.

The claims defining the invention are as follows:

1. A method of disinfection comprising the step of applying ultrasonic energy at a frequency selected to be above 1.5 MHz to a liquid composition comprising a disinfectant in combination with at least one surfactant to produce a nebulised disinfectant product.

2. A method according to claim 1 wherein the liquid disinfectant composition is selected and the ultrasound energy is applied so that 90% of microdroplets are less than 2.0 micrometers in diameter.

3. A method according to claim 1 or claim 2 wherein the ultrasonic energy is applied to the liquid composition in a nebulising chamber.

4. A method according to any one of the preceeding claims wherein the ultrasonic energy is applied for a nebulising duration and at an ultrasonic frequency selected in combination to provide a predetermined level of disinfection of an object exposed to the nebulised disinfectant product.

5. A method according to any one of the preceeding claims wherein the ultrasound frequency is above 2 MHz

6. A method according to any one of the preceding claims wherein the disinfection occurs at below 40° C.

7. A method according to any one of the preceeding claims wherein the surfactant modifies the size of the microdroplets.

8. A method according to any one of the preceeding claims wherein the surfactant modifies the susceptibility to activation of the microdroplets.

9. A method according to any one of the preceding claims wherein the disinfectant is activated by high frequency ultrasound.

10. A method according to any one of the preceding claims wherein the disinfectant is selected from the group consisting of peroxy compounds, halogenated compounds, phenolic compounds, and halogenated phenolic compounds.

11. A method according to claim 10 wherein a peroxy compound is selected from the group consisting of hydrogen peroxide, peracetic acid, persulfates, and percarbonates.

12. A method according to claim 10 wherein the disinfectant is a halogenated compound selected from sodium hydrochloride and povidone iodine.

13. A method according to claim 10 wherein the disinfectant is is Triclosan.

14. A method according to any one of the preceeding claims wherein the application of ultrasound nebulises the liquid composition within an enclosed ultrasonic chamber which resides in or communicates with an enclosed disinfection chamber.

15. A method of disinfection according to any one of the preceding claims wherein the liquid composition includes an alcohol as a surfactant

16. A method according to any one of the preceding claims wherein the nebulisation duration and ultrasonic frequency are selected such that a disinfected object is quickly dried.

17. A method of performing disinfection according to claim 15 or claim 16 wherein the disinfected article is blow dried.

18. A method of performing disinfection according to any one of the preceding claims wherein the liquid composition includes at least one substance with a high vapour pressure relative to water.

19. A method according to claim 18 wherein the at least one substance with high vapour pressure is selected to reduce drying time.

20. A method according to any one of claims 18 to 19 wherein the at least one substance with high vapour pressure is selected from the group consisting of alcohols, ethers, hydrocarbons, and esters.

21. A method according to any one of the preceding claims further including the step of neutralising the disinfectant with a neutralising agent subsequent to the disinfection step.

22. A method according to claim 21 wherein the neutralising agent is applied in nebulised form.

23. A method according to claim 21 or 22 wherein the neutralising agent is selected from the group consisting of peroxidase enzymes or sodium thiosulfate.

24. A disinfected volume in a nebulising chamber prepared by a method according to any one of the proceeding claims.

25. A composition for use in a disinfection method according to any one of the preceding claims comprising a disinfectant in combination with a surfactant.

26. A composition according to claim 25 wherein the disinfectant is selected from the group consisting of peroxy compounds, halo compounds, phenolic compounds, and halogenated phenolic compounds.

27. A composition according to claim 26 wherein the disinfectant is selected from the group consisting of hydrogen peroxide, peracetic acid, persulfates, and percarbonates.

28. A composition according to claim 26 wherein the disinfectant is selected from sodium hydrochloride and povidone iodine.

29. A composition according to claim 26 wherein the disinfectant is Triclosan.

30. A composition according to any one of claims 25 to 29 further comprising a surfactant.

31. A composition according to any one of claims 25-29 wherein the surfactant is one or more compounds selected from the group consisting of ethoxylated alcohols, dodecylbenzene sulfonic acid salts, block copolymers of ethylene oxide and propylene oxide and alcohol.

32. A composition according to claim 31 wherein the surfactant is Teric 12A3.

33. A composition according to any one of claims 25 to 32 further comprising a substance with a higher vapour pressure than water.

34. A composition according to claim 33 wherein the substance and/or mixture of substances with higher vapour pressure is selected from the group consisting of alcohols, ethers, hydrocarbons, and esters.

35. A mist comprising droplets of a composition containing a disinfectant and having 90% of the droplets between 0.8 and 2.0 micrometres in diameter when formed by the method of any one of claims 1 to 23.

36. A mist according to claim 35 when formed from the nebulisation of a composition according to any one of claims 25 to 34.

37. A disinfected article when disinfected according to a method of any one of claims 1 to 23, or by exposure to a mist according to claim 35 or 36.

38. A disinfected article according to claim 37 in the form of a dental impression.

39. A method of disinfection comprising the step of applying ultrasonic energy at a frequency selected to be above 1.5 MHz to a nebulised composition comprising a disinfectant in combination with at least one surfactant.

40. A method of disinfection comprising the step of nebulising a liquid disinfectant in combination with at least one surfactant to form microdroplets, allowing the microdroplets to contact a surface and applying ultrasonic energy to at least one of the surface and the microdroplets.

41. Apparatus for disinfection comprising;
a closed disinfection chamber adapted to receive an article to be disinfected;
a nebulizer comprising a nebulizing chamber adapted in use to receive a disinfecting agent to be nebulised, said nebuliser having an outlet for discharging a nebulised disinfecting agent directly and without intermediate tubing into the closed disinfection chamber, and having an intake communicating directly and without intermediate tubing with the disinfection chamber interior; and
a transducer adapted to sonicate the disinfecting agent within the nebulising chamber;
whereby in use air entering the nebulising chamber via said intake carries a progressively increasing concentration of nebulised disinfectant.

42. Apparatus according to claim 41 wherein the nebuliser is situated wholly or partly within the disinfection chamber.

43. Apparatus according to claim 41 or 42 wherein the transducer is exterior of the disinfection chamber.

44. Apparatus according to any one of claims 41 to 43 wherein the transducer is adapted to sonicate the disinfectant at a frequency of 1 MHz or greater.

45. Apparatus according to any one of claims 41 to 44 wherein the nebulising chamber receives hydrogen peroxide or a compound containing hydrogen peroxide.

46. Apparatus according to any one of claims 41 to 46 wherein ingress of air is excluded from the apparatus during sonication of the disinfectant.

47. Apparatus according to any one of claims 41 to 46 wherein the nebuliser is of a type in which the transducer creates an ultrasonic fountain which nebulises the disinfecting agent and recirculates the nebulised disinfecting agent.
